(19) United States
(12) Patent Application Publication
(10) Pub. No.: US 2025/0000373 A1
(43) Pub. Date: Jan. 2, 2025

(54) Using Data from a Body Worn Sensor to Modify Monitored Physiological Data

(71) Applicant: Spacelabs Healthcare L.L.C., Snoqualmie, WA (US)

(72) Inventors: Richard Gagliardo, Sammamish, WA (US); Jeffrey Jay Gilham, Sammamish, WA (US); William Gregory Downs, Snoqualmie, WA (US)

(21) Appl. No.: 18/807,066

(22) Filed: Aug. 16, 2024

Related U.S. Application Data
(63) Continuation of application No. 16/912,685, filed on Jun. 25, 2020, now Pat. No. 12,102,416.
(60) Provisional application No. 62/866,621, filed on Jun. 26, 2019.

(51) Int. Cl. A61B 5/0205; A61B 5/00; A61B 5/11; A61B 5/25

(52) U.S. Cl. CPC A61B 5/0205; A61B 5/11; A61B 5/25; A61B 5/6801; A61B 2560/0214; A61B 2562/0219; A61B 2562/166; A61B 2562/227

(57) ABSTRACT

The present specification describes methods and systems for monitoring changes in physiological data, such as electrocardiogram data, respiration data, and blood pressure data, as a consequence of a change in position or movement of a subject under observation. Embodiments of the present specification provide systems for detecting and processing motion data by utilizing an available physiological monitoring device, with minimal additions of cost and equipment. A connecting wire is used to add a motion sensor to an existing physiological monitoring device. The connecting wire provides a channel for powering the motion sensing device as well as communication of data to and from the motion sensing device. Preferably, the motion sensor is embedded into the wire.

### CROSS-REFERENCE

The present application is a continuation application of U.S. patent application Ser. No. 16/912,685, titled “Using Data from a Body Worn Sensor to Modify Monitored Physiological Data” and filed on Jun. 25, 2020, which relies on U.S. Patent Provisional Application No. 62/866,621, of the same title and filed on Jun. 26, 2019, for priority. Both of the above referenced applications are herein incorporated by reference in their entirety.

### FIELD

The present specification relates generally to monitoring health-related parameters and more specifically to methods and systems for mounting sensors, such as a motion sensor, on the body of a person and using the sensors to provide activity and relative positional information about the person in order to correct, adjust, or otherwise modify physiological data. Additionally, the present specification relates to using a single-wire communication system for integrating the motion sensor with at least one other sensor.

### BACKGROUND

Most monitors measure irregular heart-beats or any other irregular or abnormal physiological activity. An ambulatory electrocardiogram (AECG), which is consistently worn anywhere between 24 hours to a week or more, monitors electrocardiogram (ECG) data. Similarly, blood pressure (BP) monitors are used for hypertension management and cardiac monitoring. The monitors generate alarms, which may indicate an, or varying levels of, emergency in response to detection of an abnormal condition. However, often there are cases when a sensor detects what appears to be abnormal physiological activity due to a change in the patient's position or due to the patient's movement, yet the patient is actually healthy and his or her health status does not warrant an alarm. This may be especially true for ambulatory patients. For example, a person using a BP monitor may be exercising when the BP levels are detected as being abnormal. Similarly, an AECG monitor may falsely raise an alarm when the heart-beat of a wearer appears to be abnormal during exercising.

Even though exercise may skew physiological monitoring, low acuity patients need to be active to speed their recovery. Therefore, it may be desirable to be able to monitor their movements over a period of time. Some monitors combine information about different types of physiological data to conclude whether a wearer of the monitor(s) (or patient) is experiencing an abnormal health condition. For example, several models of BP monitors from various manufacturers have been developed with an added function of irregular heartbeat detection. However, these monitors are also prone to providing false positives when an otherwise healthy person is in motion. In addition to exercising, false positives may also be generated due to other external events, such as any other type of physical stress for example while lifting an object, work, fatigue, and changes in environmental conditions. Sometimes, even changing posture while sleeping may generate a false positive.

There is therefore a need to combine motion detection information, such as through motion sensors, to be able to effectively monitor physiological data and reduce or eliminate false positives generated by physiological monitors. There is also a need to correlate motion and/or positional information of a patient with any other physiological data, which may be monitored continuously, regularly, or in real time, so as to enhance the reliability and accuracy of physiological data monitors and improve diagnosis. Current physiological monitoring systems, such as AECG monitors, are unable to effectively integrate motion detection information. The monitoring systems fail to effectively combine a motion detector within existing components without having to introduce circuit-level changes or other forms of system-related modifications. Therefore, there is a need for a simple method and system that may be seamlessly integrated with existing monitoring systems, to add the capability of motion detection. It is also desirable to combine motion detection information with the physiological monitoring information to provide users with correlated data.

Many communication methods exist where one electronic device can communicate to another or several other devices over multiple wires. Communication methods are also needed to combine motion sensor data with devices for other purposes, in order to minimize the cost and apparatus needed for the combination. For some designs, however, it becomes more practical to minimize the number of wires necessary for communication. Devices are known to use a single-wire bus for bi-directional communication. The single wire connection used for bi-directional communication can interconnect two or more devices. A master device is known to be connected to one or more slave devices for communication of data and for slave device(s) to draw power from the master device. A system is needed that enables efficient, low-cost, and reliable communication between a motion detecting device and any other physiological monitoring device.

### SUMMARY

The following embodiments and aspects thereof are described and illustrated in conjunction with systems, tools and methods, which are meant to be exemplary and illustrative, not limiting in scope.

The present specification discloses a physiological lead wire configured to monitor a motion of a person and to monitor a physiological parameter of the person, comprising: a connecting wire having a first end and an opposing second end; a connector plug attached to the first end, wherein the connector plug is configured to electrically connect the physiological lead wire with a physiological monitoring system; a receptacle at the second end, wherein the receptacle is configured to attach to the person; a motion detector integrated into the receptacle, wherein the motion detector is configured to acquire positional and movement information of the person and transmit the positional and movement information over the connecting wire; and a physiological sensor integrated into the receptacle, wherein the physiological sensor is configured to acquire physiological data of the person and transmit the physiological data over the connecting wire and wherein the physiological data comprises at least one of ECG data, respiration data, SpO₂ data, or blood pressure data.

Optionally, the connecting wire is further adapted to channel power to the motion detector and to transmit data to and from the motion detector.

Optionally, the physiological sensor comprises an electrode configured to detect electrical signals generated by the person's cardiac activity. The electrode may be partially exposed outside the receptacle. The electrode may be configured to transmit the electrical signals generated by the person's cardiac activity through the connecting wire. Optionally, the electrode is positioned adjacent the motion detector and the motion detector comprises a printed circuit board having a power converter, a processor, a comparator and at least one of a three-axis accelerometer, a combination of a three-axis accelerometer and a gyroscope, or a combination of a three-axis accelerometer, a gyroscope, and a magnetometer integrated therein.

The present specification also discloses an electrocardiogram monitoring system configured to monitor a motion of a person and to monitor electrical signals generated by the person's heart, comprising: a monitoring device configured to receive data indicative of the electrical signals and data indicative of the motion of the person, wherein the monitoring device comprises at least two ports; a first electrocardiogram lead wire having a first end with a connector configured to connect to either of the at least two ports and having a second end with a receptacle, wherein the receptacle is configured to attach to the person and wherein the receptacle comprises an electrode and does not comprise a motion detector; and a second electrocardiogram lead wire having a first end with a connector configured to connect to either of the at least two ports and having a second end with a receptacle, wherein the receptacle is configured to attach to the person, wherein the receptacle comprises an electrode and a motion detector, and wherein the motion detector is configured to acquire positional and movement information of the person and transmit the positional and movement information over the second electrocardiogram lead wire.

Optionally, the second electrocardiogram lead wire is further adapted to channel power to the motion detector and to transmit data to and from the motion detector.

Optionally, in the first electrocardiogram lead wire, the electrode is partially exposed through the receptacle, is configured to detect the electrical signals, and is in electrical communication with the first electrocardiogram lead wire and, in the second electrocardiogram lead wire, the electrode is partially exposed through the receptacle, is configured to detect the electrical signals, and is in electrical communication with the first electrocardiogram lead wire.

Optionally, in the second electrocardiogram lead wire, the electrode is positioned adjacent the motion detector and the motion detector comprises a printed circuit board having a power converter, a processor, a comparator and at least one of a three-axis accelerometer, a combination of a three-axis accelerometer and a gyroscope, or a combination of a three-axis accelerometer, a gyroscope, and a magnetometer integrated therein.

Optionally, the monitoring device comprises a third port. Optionally, the electrocardiogram monitoring system further comprises a third electrocardiogram lead wire having a first end with a connector configured to connect to either of the at least two ports or the third port and having a second end with a receptacle, wherein the receptacle is configured to attach to the person and wherein the receptacle comprises an electrode and does not comprise a motion detector. Optionally, the monitoring device comprises a fourth port. Optionally, the electrocardiogram monitoring system further comprises a fourth electrocardiogram lead wire having a first end with a connector configured to connect to either of the at least two ports, the third port or the fourth port and having a second end with a receptacle, wherein the receptacle is configured to attach to the person and wherein the receptacle comprises an electrode and does not comprise a motion detector. Each of the at least two ports, the third port and the fourth port may be structurally equivalent and configured to receive a same shaped connector.

The present specification also discloses a respiration monitoring system configured to monitor a motion of a person and to monitor electrical signals generated by the person's respiration, comprising: a monitoring device configured to receive data indicative of the electrical signals and data indicative of the motion of the person, wherein the monitoring device comprises at least two ports; a first wire having a first end with a connector configured to connect to either of the at least two ports and having a second end with a receptacle, wherein the receptacle is configured to attach to the person and wherein the receptacle comprises a respiratory sensor and does not comprise a motion detector; and a second wire having a first end with a connector configured to connect to either of the at least two ports and having a second end with a receptacle, wherein the receptacle is configured to attach to the person, wherein the receptacle comprises a respiratory sensor and a motion detector, and wherein the motion detector is configured to acquire positional and movement information of the person and transmit the positional and movement information over the second wire.

Optionally, the second wire is further adapted to channel power to the motion detector and to transmit data to and from the motion detector.

The present specification also discloses an oxygen saturation monitoring system configured to monitor a motion of a person and to monitor electrical signals generated by the person's oxygen saturation, comprising: a monitoring device configured to receive data indicative of the electrical signals and data indicative of the motion of the person, wherein the monitoring device comprises at least two ports; a first wire having a first end with a connector configured to connect to either of the at least two ports and having a second end with a receptacle, wherein the receptacle is configured to attach to the person and wherein the receptacle comprises a blood oxygenation sensor and does not comprise a motion detector; and a second wire having a first end with a connector configured to connect to either of the at least two ports and having a second end with a receptacle, wherein the receptacle is configured to attach to the person, wherein the receptacle comprises a blood oxygenation sensor and a motion detector, and wherein the motion detector is configured to acquire positional and movement information of the person and transmit the positional and movement information over the second wire.

Optionally, the second wire is further adapted to channel power to the motion detector and to transmit data to and from the motion detector.

The present specification also discloses a method for monitoring a motion of a person and electrical signals generated by the person's heart, comprising: acquiring a monitoring device configured to receive data indicative of the electrical signals and data indicative of the motion of the person, wherein the monitoring device comprises at least two ports; connecting a first electrocardiogram lead wire to either of the at least two ports, wherein the first electrocardiogram lead wire comprises a first end with a connector configured to connect to either of the at least two ports and a second end with a receptacle, wherein the receptacle is configured to attach to the person and wherein the receptacle comprises an electrode and does not comprise a motion detector; attaching the electrode of first electrocardiogram lead wire to the person; connecting a second electrocardiogram lead wire to either of the at least two ports, wherein the second electrocardiogram lead wire has a first end with a connector configured to connect to either of the at least two ports and a second end with a receptacle, wherein the receptacle is configured to attach to the person, wherein the receptacle comprises an electrode and a motion detector, and wherein the motion detector is configured to acquire positional and movement information of the person and transmit the positional and movement information over the second electrocardiogram lead wire; attaching the electrode of second electrocardiogram lead wire to the person; activating the monitoring device; and recording data indicative of the electrical signals and data indicative of the motion of the person.

Optionally, the second electrocardiogram lead wire is further adapted to channel power to the motion detector and to transmit data to and from the motion detector.

Optionally, in the second electrocardiogram lead wire, the electrode is positioned adjacent the motion detector and wherein the motion detector comprises a printed circuit board having a power converter, a processor, a comparator and at least one of a three-axis accelerometer, a combination of a three-axis accelerometer and a gyroscope, or a combination of a three-axis accelerometer, a gyroscope, and a magnetometer integrated therein.

Optionally, the method further comprises connecting a third electrocardiogram lead wire to either of the at least two ports or a third port, wherein the third electrocardiogram lead wire has a first end with a connector configured to connect to either of the at least two ports or the third port and a second end with a receptacle, wherein the receptacle is configured to attach to the person, wherein the receptacle comprises an electrode and does not comprise a motion detector and attaching the electrode of the third electrocardiogram lead wire to the person. Optionally, the method further comprises connecting a fourth electrocardiogram lead wire to either of the at least two ports, the third port, or a fourth port, wherein the fourth electrocardiogram lead wire has a first end with a connector configured to connect to either of the at least two ports, the third port or the fourth port, and a second end with a receptacle, wherein the receptacle is configured to attach to the person, wherein the receptacle comprises an electrode and does not comprise a motion detector and attaching the electrode of the fourth electrocardiogram lead wire to the person. Each of the at least two ports, the third port and the fourth port may be structurally equivalent and configured to receive a same shaped connector.

The present specification also discloses a physiological monitoring system for monitoring a wearer of the system, comprising: at least one connecting wire, wherein each connecting wire comprises: a plug at a first end of the connecting wire; a receptacle at a second end of the connecting wire, wherein the first end is opposite to the second end of the connecting wire; and a motion sensor system in proximity to the receptacle, wherein the motion sensor system senses positional and movement information of the wearer and sends the information over the connecting wire; wherein the connecting wire provides a channel for power to power the motion sensor system and data communication to and from the motion sensor system.

Optionally, the plug comprises an interface to connect with a power source.

Optionally, the plug comprises an interface to connect with a data storage and processing system.

Optionally, the receptacle is a snap-connector receptacle that attaches with a body of the wearer.

Optionally, the receptacle attaches to a body of the wearer similarly to an Electrocardiogram (ECG) lead snap. The plug may interface with a physiological monitoring device. The physiological monitoring device may be an ECG monitoring device.

Optionally, the motion sensor system comprises: at least one motion sensor to detect the positional and movement information; and a processor to receive and process the detected information. The motion sensor may comprise at least one of a three-axis accelerometer; a combination of a three-axis accelerometer and a gyroscope; and a combination of a three-axis accelerometer, a gyroscope, and a magnetometer. The motion sensor may comprise an inclination detector. The motion sensor may be configured to provide movement indications in all directions. The motion sensor may be configured to provide angle indications in two axes.

Optionally, the motion sensor system communicates data to and from at least one other physiological monitoring device. The at least one other physiological monitoring device may be an ECG monitoring device, a respiration monitoring device, a blood pressure (BP) monitoring device, or a combination of two or more of an ECG monitoring device, a respiration monitoring device, and a blood pressure (BP) monitoring device.

The present specification also discloses a physiological monitoring system for monitoring a wearer of the system, comprising: at least one connecting wire, wherein each connecting wire comprises: a plug at a first end of the connecting wire; a receptacle at a second end of the connecting wire, wherein the first end is opposite to the second end of the connecting wire; and a motion sensor system in proximity to the receptacle, wherein the motion sensor system senses positional and movement information of the wearer and sends the information over the connecting wire; wherein the connecting wire provides a channel for power to power the motion sensor system and data communication to and from the motion sensor system.

Optionally, the plug comprises an interface to connect with a power source. Optionally, the plug comprises an interface to connect with a data storage and processing system.

Optionally, the receptacle is a snap-connector receptacle that attaches with a body of the wearer. Optionally, the receptacle attaches to a body of the wearer similarly to an Electrocardiogram (ECG) lead snap.

Optionally, the plug interfaces with a physiological monitoring device. The physiological monitoring device may be an ECG monitoring device.

Optionally, the motion sensor system comprises: at least one motion sensor to detect the positional and movement information; and a processor to receive and process the detected information. The motion sensor may comprise at least one of a three-axis accelerometer; a combination of a three-axis accelerometer and a gyroscope; and a combination of a three-axis accelerometer, a gyroscope, and a magnetometer. The motion sensor may comprise an inclination detector. The motion sensor may be configured to provide movement indications in all directions.

The motion sensor may be configured to provide angle indications in two axes.

Optionally, the motion sensor system communicates data to and from at least one other physiological monitoring device. The at least one other physiological monitoring device may be an ECG monitoring device, a respiration monitoring device, a blood pressure (BP) monitoring device, or a combination of two or more of an ECG monitoring device, a respiration monitoring device, and a blood pressure (BP) monitoring device.

The present specification also discloses a physiological monitoring system for monitoring a wearer of the system, comprising: at least one connecting wire, wherein each connecting wire comprises: a plug at a first end of the connecting wire, wherein the plug is configured to interface with a receptacle on the physiological monitoring system; a housing at a second end of the connecting wire, wherein the first end is opposite to the second end of the connecting wire; and a motion sensor system in the housing, wherein the motion sensor system senses positional and movement information of the wearer and sends the information over the connecting wire, wherein the motion sensor system comprises a power converter for converting power from the power source. The connecting wire provides a channel to transmit power from a power source in the physiological monitoring system to the motion sensor system and communicate positional and movement information from the motion sensor system.

Optionally, the receptacle on the physiological monitoring system is one of a plurality of receptacles. A shape of the receptacle may be different from the rest of the plurality of receptacles, or may be same as the rest of the plurality of receptacles.

The aforementioned and other embodiments of the present specification shall be described in greater depth in the drawings and detailed description provided below.

### BRIEF DESCRIPTION OF THE DRAWINGS

These and other features and advantages of the present specification will be appreciated, as they become better understood by reference to the following detailed description when considered in connection with the accompanying drawings, wherein:

FIG. 1A illustrates a connecting wire comprising a motion sensor, in accordance with some embodiments of the present specification;

FIG. 1B illustrates an adapter cable to connect a connecting wire of FIG. 1A, in accordance with some embodiments of the present specification;

FIG. 1C illustrates an alternative embodiment of a physiological monitoring device comprising a separate connector for interfacing a motion sensing system, in accordance with some embodiments of the present specification;

FIG. 1D is a flow chart showing steps for an exemplary process for monitoring both a motion of a person and electrical signals generated by the person's heart, in accordance with some embodiments of the present specification;

FIG. 2 illustrates an orthographic view of a receptacle shown in FIG. 1, in accordance with some embodiments of the present specification;

FIG. 3A illustrates a first portion of a PCB configured to carry components of motion sensor system shown in FIG. 2, in accordance with some embodiments of the present specification;

FIG. 3B illustrates a second portion of a PCB, which is on a side opposite to the side of first portion, configured to carry components of motion sensor system shown in FIG. 2, in accordance with some embodiments of the present specification;

FIG. 4 is a block diagram illustrating components of two devices that share power and communication over a single wire, in accordance with some embodiments of the present specification;

FIG. 5 is an exemplary simulation circuit that implements single wire communication in accordance with some embodiments of the present specification;

FIG. 6A illustrates a sample of data transmission from a first device to a second device using the simulation circuit of FIG. 5, in accordance with some embodiments of the present specification;

FIG. 6B illustrates a sample of data transmission from the second device to the first device using the simulation circuit of FIG. 5, in accordance with some embodiments of the present specification;

FIG. 7A is a photograph of a plug portion of a wire, in accordance with some embodiments of the present specification;

FIG. 7B is a line drawing of the plug portion of the wire shown in FIG. 7A, including a cross-sectional view of its housing;

FIG. 7C is a schematic drawing of the plug portion of the wire, shown in FIG. 7A, along with dimensions of its components;

FIG. 8A illustrates a top view of a receptacle portion of a wire, in accordance with some embodiments of the present specification;

FIG. 8B illustrates a side view of the receptacle portion of the wire, shown in FIG. 8A;

FIG. 8C illustrates a cross-sectional side view of the receptacle portion of the wire, shown in FIG. 8A; and

FIG. 8D illustrates a bottom view of the receptacle portion of the wire, shown in FIG. 8A.

### DETAILED DESCRIPTION

In various embodiments, the present specification provides methods and systems to seamlessly integrate a motion detection system with existing physiological monitoring system. The motion detection system monitors changes in position and/or movement of a wearer of the physiological monitoring system. The monitored changes may be correlated with other physiological monitoring data to identify physiological abnormalities and aid in improving diagnosis. A single wire communication system enables interfacing between the position and/or movement sensing device and an existing or conventional physiological monitor.

Embodiments of the present specification provide a motion sensor system that can be embedded within a connecting wire that has the form and structure of an ECG lead wire. The connecting wire is compatible with a monitoring device, such as an ECG monitoring device. The connecting wire is connected similar to and in addition to other ECG lead wires that measure cardiac signals, to a physiological monitoring device. The connecting wire is used for providing power to the motion sensor system, which is integrated into the distal body of the ECG lead wire, and supporting bi-directional communication between the motion sensor system and the monitoring device. In alternative embodiments, the connecting wire with motion sensor system is compatible with any other physiological monitoring device, in addition to an ECG monitoring device. In embodiments, the motion sensor information is combined with information from one or more other physiological sensors to identify abnormalities and improve diagnosis.

The present specification is directed towards multiple embodiments. The following disclosure is provided in order to enable a person having ordinary skill in the art to practice the invention. Language used in this specification should not be interpreted as a general disavowal of any one specific embodiment or used to limit the claims beyond the meaning of the terms used therein. The general principles defined herein may be applied to other embodiments and applications without departing from the spirit and scope of the invention. Also, the terminology and phraseology used is for the purpose of describing exemplary embodiments and should not be considered limiting. Thus, the present invention is to be accorded the widest scope encompassing numerous alternatives, modifications and equivalents consistent with the principles and features disclosed. For purpose of clarity, details relating to technical material that is known in the technical fields related to the invention have not been described in detail so as not to unnecessarily obscure the present invention.

In the description and claims of the application, each of the words “comprise” “include” and “have”, and forms thereof, are not necessarily limited to members in a list with which the words may be associated. It should be noted herein that any feature or component described in association with a specific embodiment may be used and implemented with any other embodiment unless clearly indicated otherwise.

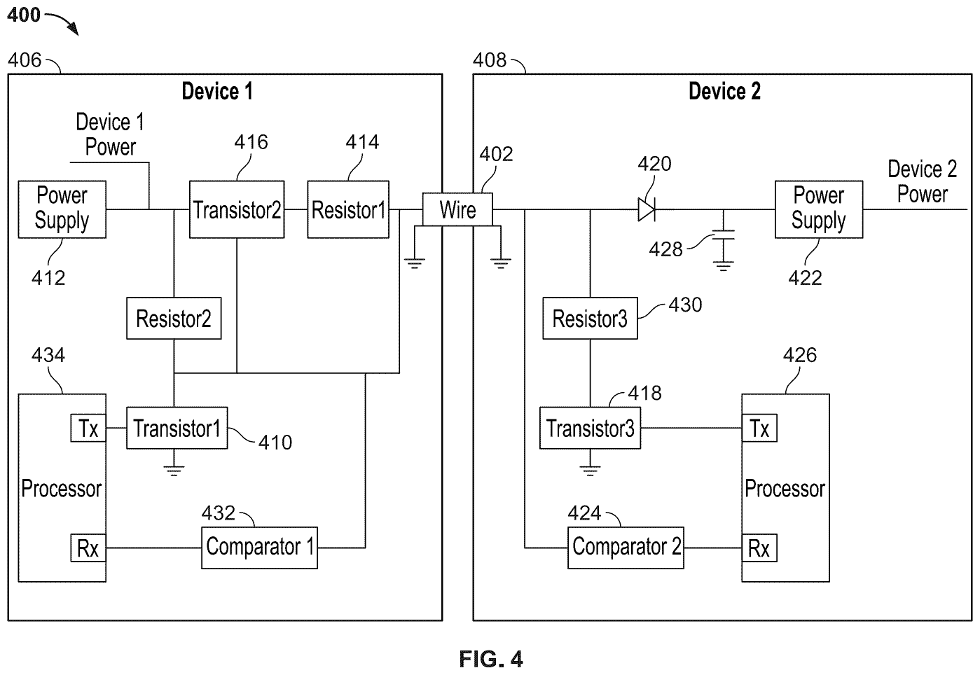

Embodiments of the present specification provide a connecting wire capable of connecting to a physiological monitoring device, such as an ECG monitor, also termed herein as ECG device or ECG monitoring device. Embodiments of the connecting wire are described below with respect to FIGS. 4, 5, 6A, and 6B. In an embodiment, the ECG device is a system that senses and analyses ECG signals by recording electrical activity of the heart. The monitoring is performed over a period of time using electrodes that are placed on the skin of a subject/person, such as a patient or other individual. In embodiments, the person is a patient or any other living being that is under observation for monitoring by the systems of the present specification. The ECG device typically interfaces with the electrodes through a connecting lead wire (ECG lead wire). The lead wire comprises an attachment mechanism at one end (proximal) for connection to the electrode(s) that are positioned on the subject's skin. The opposite end (distal) of the lead wire comprises a plug that interfaces with the ECG device. The heart muscle's electrophysiological pattern of depolarizing and repolarizing is measured and viewed in the form of a graph of voltage versus time (electrocardiogram). The electrocardiogram can be viewed on a screen attached to the ECG device, and/or can be printed on paper.

Ambulatory ECG devices use a small monitoring device worn by the subject which transmits monitored data from the device to a distant monitoring station using wireless communication. The device itself records, analyses, and communicates ECG data. Hardware components in the device enable sensing and storage, while software elements enable processing of data.

Sometimes hemodynamic monitoring is performed simultaneously with cardiac monitoring. Hemodynamic monitoring is usually performed using hydraulic circuits that monitor properties of blood flow. Some monitors combine respiration monitoring with ECG monitoring and/or hemodynamic monitoring, or just blood pressure (BP) monitoring. Respiration monitoring devices indicate respiration data like respiration rate, amplitude, and other characteristics. Most of these and other physiological monitoring devices receive the data concerning their objective, but also tend to receive noise that may arise due to motion of the subject. The motion data, when combined with other physiological monitoring data provides crucial diagnostic information about the subject.

Embodiments of the present specification can be configured to interface with an ECG device, a respiration monitoring device, a BP monitoring device, any other physiological monitoring device, or a combination of two or more of these devices. For instance, embodiments could be used on a patient who is wearing a Non-Invasive BP (NIBP) cuff or an SpO₂ sensor. The motion information derived from embodiments of the present specification would be used to provide more context to the collected NIBP or SpO₂ data, such as whether the patient is sitting up or active at the time of the reading. While some embodiments of systems of the present specification are described in the context of an ECG device (as the systems attach similarly to ECG electrodes and can be attached to a monitor in the same manner as an ECG lead wire, such as through a combiner (Yoke) cable or directly to the monitor), the systems of the present specification do not rely on any of the ECG components to operate. Embodiments of the present specification provide a low cost, portable option to additionally monitor position and movement-related data of a subject over a single wire for power and data, and combine the motion data with the other physiological data, in order to improve medical diagnosis as well as determine health or fitness levels.

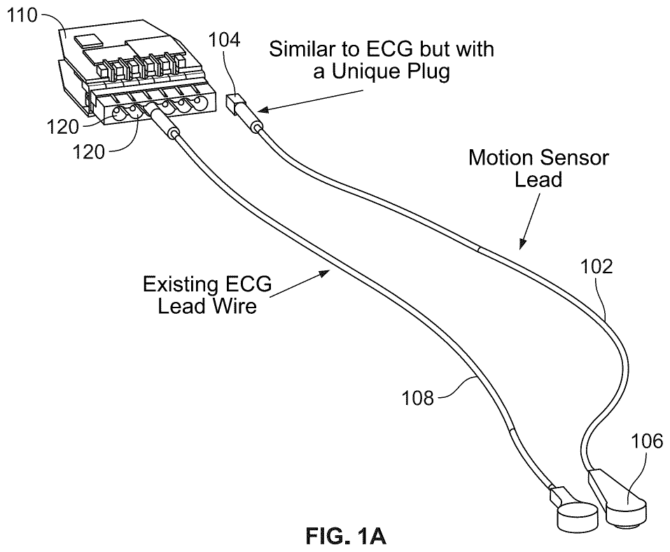

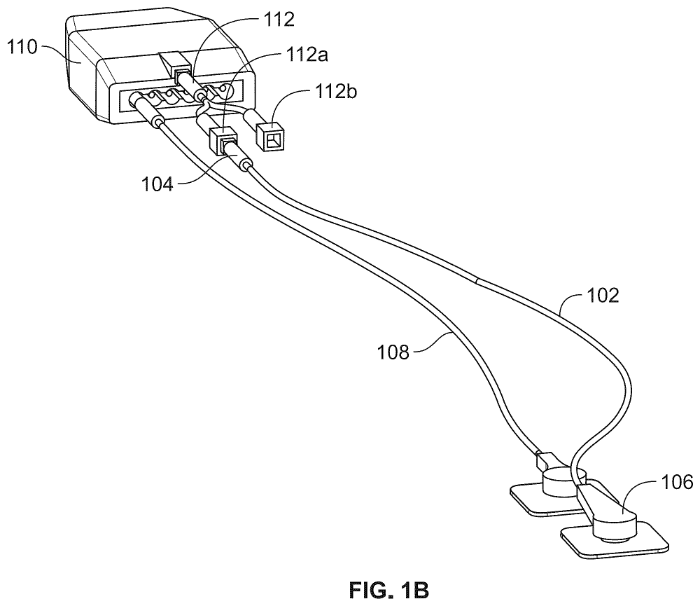

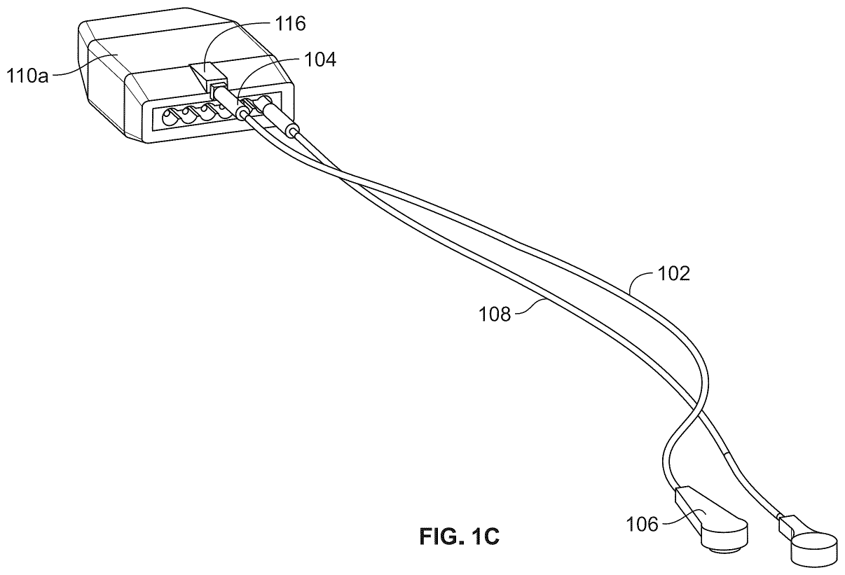

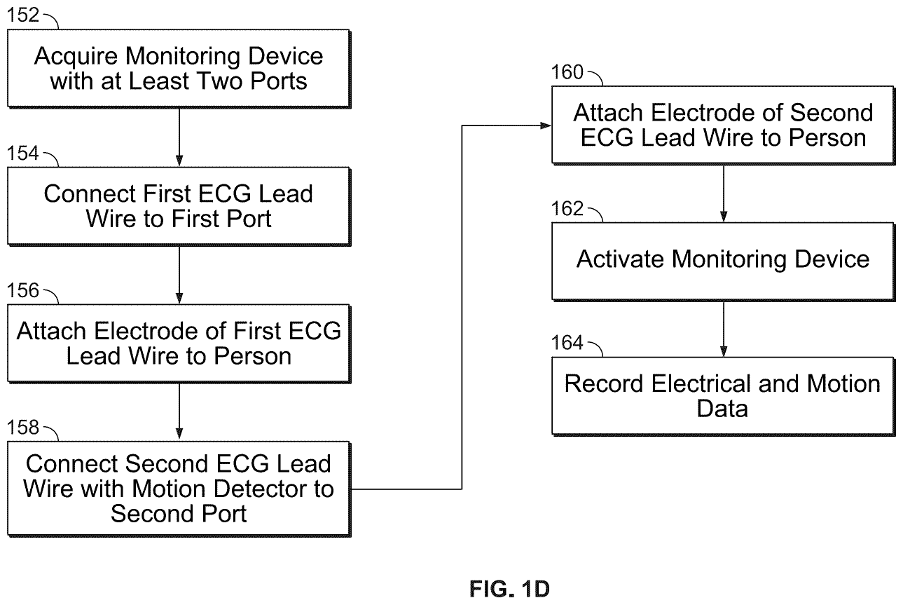

FIG. 1A illustrates a connecting wire 102 comprising a motion sensing system, in accordance with some embodiments of the present specification. Wire 102 has two opposing ends including a connector/plug 104 at a first (distal) end and a receptacle 106 at a second (proximal) end. In one implementation, plug 104 is similar to the plug of an ECG lead wire 108 and therefore compatible with a conventional ECG monitor. Additionally, plug 104 can interface with a device 110. In one embodiment device 110 is an ECG device, and plug 104 connects to the ECG device similarly to the manner in which an ECG lead wire 108 interfaces with device 110. Accordingly, the present invention is directed toward a lead wire having a connector at one end that is compatible with a connection port of a conventional ECG monitoring device, a connection port of a conventional respiration monitoring device, a connection port of a conventional SpO₂ monitoring device, or a connection port of a conventional BP monitoring device such that the connector is structurally similar to a connector of a conventional ECG lead wire, a connector of a conventional respiration sensor, a connector of a conventional SpO₂ sensor, or a connector of a conventional blood pressure cuff, none of which have a motion sensor integrated therein.

The electrocardiogram monitoring system of FIG. 1A, according to embodiments of the present specification is configured to monitor a motion of a person and to monitor electrical signals generated by the person's heart. The ECG monitoring device 110 receives data indicative of the electrical signals from one or more ECG lead wires, including but not limited to lead wire 108 and motion sensor lead 102. Additionally, the motion sensor lead 102 provides data indicative of the motion of the person. Device 110 includes multiple ports 120 where at least one of the ports is used to connect to the motion sensor lead 102. The receptacle 106, positioned at the second (proximal) end of the motion sensor lead 102, attaches to the patient and includes an electrode and a motion detector to acquire positional and movement information of the patient and transmit the information over the motion sensor lead 102. One or more of the other remaining ports 120 on device 110 connect to one or more lead wires 108, which do not include a motion detector.

In one implementation receptacle 106 is configured similar to a snap-attach receptacle of ECG lead wire 108. A snap connector, also known as a pinch clip connector, may be attached to the body of a subject. The subject could be a patient, or any other being who is a wearer of the monitoring system and is to be monitored by the various embodiments of the present specification. Receptacle 106 may use the ECG adhesive snaps as a way to attach to the patient's body. In embodiments, the position or placement of receptacle 106 on the patient's body is independent of the placement of any ECG electrode. In some embodiments, optimal locations for placement of receptacle 106 are suggested to the patient, which allow for better detection of respiration activity (used to verify respiratory data or to signal breathing difficulty or stress).

In one embodiment, ECG adhesive pads are used to attach receptacle 106 to the subject. In various embodiments, receptacle 106 is configured in a manner similar to any type of an ECG electrode connector, such as but not limited to a wire dumbbell connector, a locking slot connector, or a keyhole connector. In embodiments, at least one motion sensor system is embedded in proximity to receptacle 106, and preferably within the housing of the receptacle 106. In one embodiment, a housing at the second end of wire 102 a motion sensor positioned within the receptacle 106. Connecting wire 102 is uniquely configured to communicate motion detection data from receptacle 106 to plug 104, which may be further recorded and/or processed by separate circuits within device 110. Connecting wire 102 provides a single path for powering the motion sensing device in receptacle 106 and enabling bi-directional communication between the motion sensing system and device 110. In embodiments, the data collected through the motion sensor system is correlated with data from an ECG monitor and/or other physiological monitoring systems, such as respiration data and blood pressure (BP) data.

In an embodiment, an adapter cable is used to connect multiple motion sensor systems to a physiological monitoring system, such as device 110 shown in FIG. 1. A plug portion of the adapter is configured to connect to a specific device 110 and may include any safety feature or unique/specialized aspect required to allow plug 104 to connect with wire 102. Multiple receptacles are electrically coupled to device 110 through the adapter cable, plug 104, and wire 102. FIG. 1B illustrates an adapter cable 112 used to connect multiple wires to device 110 of FIG. 1, in accordance with some embodiments of the present specification. Plug 104 connects to a connector 112a of adapter 112, while another connector portion 112b of adapter 112 is available to connect another wire, such as another motion sensor system. Additionally, FIG. 1C illustrates an alternative embodiment of a device 110a that includes a separate connector 116 to connect plug 104. Connector 116 may be provided in addition to the conventional connectors for interfacing with physiological monitoring device 110a. Connector 106 may also interface with an adapter 112 to connect with multiple sensors. Accordingly, connector comprises a first connector portion 112 configured to connect to a connector port of a conventional ECG monitoring device, a connection port of a conventional respiration monitoring device, or a connection port of a conventional BP monitoring device, a wire extending therefrom and being split into two or more prongs, where each prong leads to a port (112a, 112b, etc.) configured to receive a connector portion of an ECG lead wire, a cable of a respiration sensor, or a cable of a blood pressure cuff. It should be appreciated that while FIG. 1A shows a two-pronged connection, there could be 3, 4, 5, 6, 7, 8, 9, 10, or 100 prongs, or any whole number increment therein.

FIG. 1D is a flowchart showing exemplary process steps for monitoring both a motion of a person and electrical signals generated by the person's heart, in accordance with some embodiments of the present specification. The person is a patient or any other living being that is under observation for monitoring by the systems of the present specification. With reference to both FIGS. 1A and 1D, at step 152, the person, a physician or any other care provider for the person, acquires a monitoring device, such as device 110, configured to receive data indicative of the electrical signals and data indicative of the motion of the person. The monitoring device 110 includes two or more ports 120 that are used to connect ECG lead wires such as lead wire 108, which does not include a motion detector, and wire 102 which includes a motion detector, in their respective receptacles. Each of the two or more ports 120 of device 110 are structurally equivalent and are configured to receive a same shaped connector. At step 154, a first ECG lead wire, such as wire 108, is connected to a first port. In embodiments, wire 108 does not include a motion detector. The first port can be any one of the two or more ports 120 on device 110. A first end of the wire 108 includes a connector configured to connect to the port of device 110. A second end of the wire 108 includes a receptacle configured to attach to the person. The receptacle includes an electrode and does not comprise a motion detector. At step 156, the electrode of first ECG lead wire is attached to the person at a suitable location on the body of the person. At step 158, a second ECG lead wire, such as wire 102, is connected to a second port of the two or more ports 120 on device 110. The second ECG lead wire has a first end with a connector, such as plug 104, configured to the corresponding port on device 110. A second end of the wire 102 has a receptacle, such as receptacle 106, which includes an electrode and a motion detector, configured to attach to the person. The motion detector is configured to acquire positional and movement information of the person and transmit the positional and movement information over the second ECG lead wire (wire 102) to the monitoring device 110 when it is activated. Once device 110 is activated, wire 102 channels power to the motion detector and to transmit data to and from the motion detector. At step 160, the electrode of second ECG lead wire is attached to the person in a manner similar to any other ECG lead wire, such as wire 108. In some embodiments, a third ECG lead wire is attached to the device 110 at third port of the two or more ports 120. The third ECG lead wire is similar to the first ECG lead wire 108, and does not include a motion detector. A receptacle of the third wire is attached to the person similar to the first wire 108. In some embodiments, a fourth ECG lead wire is attached to the device 110 at yet another one of its ports. The fourth ECG lead wire is similar to the first ECG lead wire 108 and the third wire, and does not include a motion detector. A receptacle of the fourth wire is attached to the person similar to the first wire 108. At step 162, the monitoring device 110 is activated. The monitoring device 110 is activated by enabling power supply to operate the device 110, and optionally by selecting one or more options through a user interface such as buttons, to activate the device 110. At step 164, the monitoring device 110 records data indicative of the electrical signals and data indicative of the motion of the person.

The conventional physiological monitoring devices record and analyze data pertaining to their intended physiological parameter. Integration of real-time physiological data with position and/or movement-related data can be more effective in determining changes in the physiology of the subject. Physiological data can be monitored as a result of change in posture or movement of the subject.

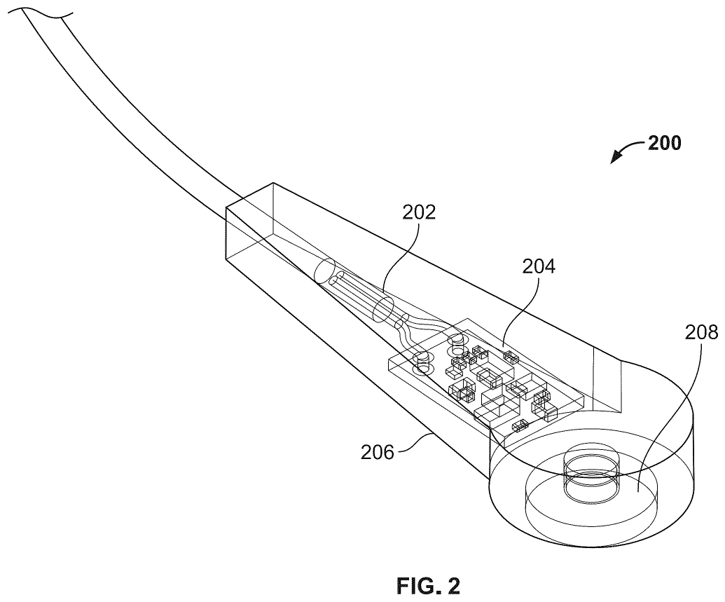

FIG. 2 illustrates an orthographic view of a receptacle 200 shown in FIG. 1, in accordance with some embodiments of the present specification. Receptacle 200 includes a housing 206 that encompasses components of an integrated motion sensor system 204. A lead connector 208 is located at the distal end of a connecting wire 202 within a portion of the housing 206 that interfaces with an electrode connector, analogous to an ECG electrode connector for the purpose of attaching receptacle 200 to the body of the subject. Motion sensor system 204 may comprise multiple components placed on a printed circuit board (PCB), and including elements that detect and process position and movement-related data. Accordingly, in one embodiment, the present invention is directed to a ECG electrode having a housing, an electrode embedded into, and exposed outside of, the housing, and a motion detector integrated into the housing, proximate the electrode.

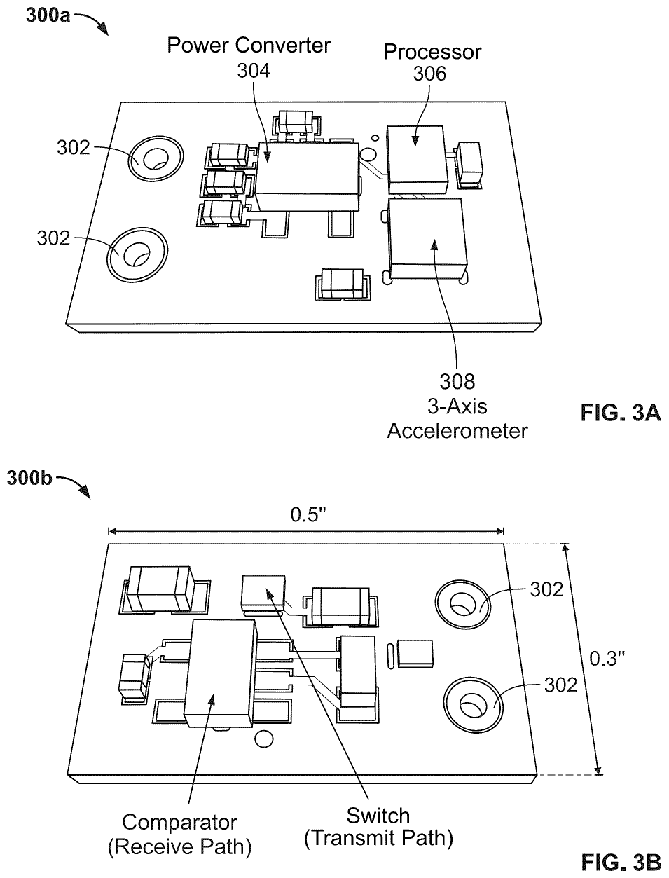

FIG. 3A illustrates a first portion 300a of a PCB configured to carry components of motion sensor system 204 shown in FIG. 2, in accordance with some embodiments of the present specification. FIG. 3B illustrates a second portion 300b of a PCB, which is on a side opposite to the side of first portion 300a, configured to carry components of motion sensor system 204 shown in FIG. 2, in accordance with some embodiments of the present specification. Referring simultaneously to FIGS. 3A and 3B, the PCB (300a, 300b) is configured to be housed within a housing of a receptacle of a lead connecting wire that couples the motion sensor system to a monitoring device that stores and processes motion sensor data alone, or in combination with other physiological monitoring data. In embodiments, the PCB (300a, 300b) is sized in order to fit within the housing of the receptacle. In one embodiment, the PCB (300a, 300b) is 0.5 inches long and 0.3 inches wide, with electrical and electronic components on both sides. In some embodiments, electrical pads 302 on the PCB (300a, 300b) are configured to solder the ground and power/communication wires to the PCB (300a, 300b).

The connecting wire may be soldered to one of the electrical pads 302 to enable communication between PCB (300a, 300b) components and a power source and physiological data monitoring device. Power from the source may be communicated over the connecting wire and received by a power converter 304. Power converter 304 is configured to drop the power on the cable down to a recommended chip voltage and to remove the fluctuation of the power due to the signalling, and thereby power the electronic components of PCB (300a, 300b). A processor 306 is configured to both process sensor data and facilitate communication to and from the physiological monitoring device. A motion sensor 308 detects position and movement-related data and provides the data to processor 306. In some embodiments, motion sensor 308 is a multi-axis accelerometer. In one embodiment, motion sensor 308 is a tri-axis accelerometer. In various embodiments, motion sensor 308 could include a ‘6-axis’ sensor (3-axis accelerometer and 3-axis gyroscope), or a ‘9-axis’ sensor (3-axis accelerometer, 3-axis gyroscope, and 3-axis magnetometer). The sensors are used to provide positional and orientation information in 3-axis, which could be used to determine an orientation of a patient, such as determining if a patient is facing down a corridor as opposed to across it. Given that an accelerometer will indicate a value of 1 G straight downward (due to gravity), the accelerometer may be used to determine inclination of a patient. Quick changes in the acceleration indicated by the accelerometer may show motion of the subject, whereas slow changes in the acceleration may indicate an inclination change (for example, sitting up or rolling on one's side). Motion sensor 308 is configured to detect at least one or more of a position, inclination, and movement of the subject.

Embodiments of the present specification can be configured to interface with different types of physiological monitoring devices, such as respiration monitoring devices, BP monitoring devices, and devices that monitor multiple physiological parameters but, in each case, are preferably positioned within the housing of a conventional physiological sensor positioned on the patient's body.

Embodiments of the present specification are used to monitor exercise data of the subject. Activity levels may be quantified to provide helpful indications about the exercises performed by the subject. For example, the number of steps can be monitored. Embodiments of the present specification may also be used to indicate a type and duration of one or more activities performed by the subject. For example, physiological data is combined with posture information to determine whether the subject is sitting, standing, awake, or asleep, for a healthy duration. Similarly, embodiments may be used to determine levels of inactivity. For example, a subject who is bed-ridden is monitored for duration(s) of inactivity and an alarm is generated to remind that the subject needs to be moved to avoid bed-sores, or if they have deceased. Embodiments can be used to also detect fall of a subject. Embodiments can also be used to detect movement by a subject that may be unwarranted, accidental, or unhealthy. For example, movement of a subject exiting the bed can be detected when they are not supposed to leave on their own. Embodiments can also be used to detect rapid movements, such as but not limited to seizures, tremors, epileptic episodes, shivers, rapid breathing due to discomfort, coughing, vomiting, and rolling in bed.

Embodiments of the present specification may combine respiration detection data with motion sensor data to monitor chest motion and detect apnea. Additionally, measurement of respiration characteristics can be suppressed during a healthy exercise regimen. Similarly, when combining BP measurement data, a monitoring attempt can be cancelled, delayed or retried at later time, if the subject is identified to be excessively active.

Embodiments of the present specification assist in minimizing false ECG ST Segment alarms, which may otherwise occur due to positional changes. Additionally, false ECG rhythm alarms, such as v-tach (ventricular tachycardia), v-run (ventricular run), or any other ECG parameter, due to changes in position or due to motion, are minimized. A combination of ECG data, respiration data, BP data, with the motion sensor data in accordance with the embodiments of the present specification can detect and raise an appropriate alarm if a subject has a critical condition, for example, if the heart rate is low and there is a decrease in pulse amplitude.

Referring now to FIG. 4, a block diagram 400 of two devices that are connected using a single wire 402 for power and data communication is illustrated in accordance with some embodiments of the present specification. In embodiments, device 406 corresponds to device 110 (FIG. 1A) used for physiological monitoring, and device 408 corresponds to an integrated motion sensor system 204 (FIG. 2) placed within a receptacle 106 (FIG. 1A). In embodiments, a first device 406 and a second device 408, are respectively configured as a master device and a slave device. Device 406 provides power to device 408, and both devices communicate with each other over wire 402. In some embodiments, multiple slave devices are connected to first device 406.

In one embodiment during normal operation, a first transistor 410 in first device 406 does not conduct, thus allowing power from a power source and through a power module 412, through a second transistor 416, and through a first resistor 414 to wire 402 and thus to any connected devices, such as device 408. First device 406 and second device 408, each have a transistor 416 and 418, respectively, which are non-conducting. The transistor 418 in second device 408 is normally non-conducting. Therefore, the power sourced by device 406 over wire 402 to device 408 flows through a diode 420 positioned between transistor 418 and a power module 422 within device 408, towards power module 422. A comparator 424 is configured to receive power sent over wire 402, within device 408. Comparator 424 compares the input power rail to a reference voltage and outputs a ‘low’ to a receiving pin on a processor 426 of device 408.

In one embodiment, for transmitting a bit from first/master device 406 to second/slave device 408, first transistor 410 is moved to a conducting state which switches transistor 416 to a non-conducting state. The side of first resistor 414 that is connected to wire 402, is pulled ‘low’ through conducting transistor 410. On the other side of wire 402, in second device 408, comparator 424 senses the input supply going ‘low’ and switches the receiving pin on processor 426 to ‘high’. In some embodiments, ‘low’ and ‘high’ signify a level of voltage, which can be interpreted by digital circuits as binary data. In some embodiments the ‘high’ and ‘low’ states can be opposite—that is what is specified in this description as a ‘low’ could be a ‘high’ and a ‘high’ could be a ‘low’. In some embodiments, the transition on the receiving pin, from ‘low’ to ‘high’ denotes a binary ‘1’. Diode 420 prevents the voltage into the power module 422 of second device 408 from dropping rapidly. A capacitor 428 positioned between the line connecting resistor 420 and power module 422, and the ground, provides a small amount of power as the power supplied to device 408 through its power module 422 starts to drain the current from that node. The combination of diode 420 and capacitor 428 momentarily minimizes the voltage drop into the power supply from power module 422.

Once a sufficient amount of time is given for second device 408 to have seen the input from wire 402 drop ‘low’, first device 406 turns first transistor 410 back to a non-conducting state, which turns transistors 416 to a conducting state, thereby allowing a normal amount of current to flow through wire 402. If first transistor 410 is turned on to a conducting state and then back to a non-conducting state relatively quickly, second device 408 registers the change as data, but the power supplied to second device 408 remains constant. The amount of time in which transistor 410 changes its state from on to off may be determined on the basis of amount of current consumed by device(s) 408, the leakage of current back through diode 420, and the size of capacitor 428.

For second device 408 to transmit a bit of data to first device 406, transistor 416 and therefore transistor 418 are turned on to a conducting state by processor 426. Input from wire 402 through a resistor 430, positioned between output of wire 402 and transistor 418 of second device 408, is momentarily pulled ‘low’. A comparator 432 on first device 406 senses the power output through resistor 414 drops to ‘low’ and consequently changes a receiving pin on a processor 434 within first device 406 to ‘high’. After a sufficient amount of time expires, processor 426 on second device 408 switches off conduction through transistor 418 and wire 402 input to second device 408 rapidly rises back up to the supply level since that input is no longer shorted to ground. Comparator 432 detects the output voltage transitioning back to ‘high’ and sets the receiving pin on processor 434 of first device 406 back to ‘low’.

In embodiments, resistor 414 on first device 406 keeps the regulator of first device 406 (the supply) from overcurrent during the intermittent short circuit events seen on the power line during transmission by second device 408. During these transmissions, transistors 416 and 410 are in a conducting state, so any momentary short circuit events of wire 402 are detected by power module 412 which puts system 400 into an overcurrent condition.

The communication over wire 402 is asynchronous, implying that a device (first device 406 or second device 408) could initiate communication on wire 402 at any time. Therefore, it is important for each device 406 and 408 to be able to detect possible data collisions. A data collision may occur when both devices 406 and 408 are sending data at the same time. When a device wishes to transmit data, it enters into a transmit state. In that state it should only detect the receiving pin change state when it has changed the state of a device processor's transmitting pin. If the receiving pin changes when the transmitting pin has not changed, then the processor (434, 426) concludes that it has sensed a collision and an upper level protocol of processors 434 and 426 of system 400 are signaled of such an event. It will be up to the upper level protocol to initiate any corrective action (usually a backoff for some random amount of time followed by a retry).

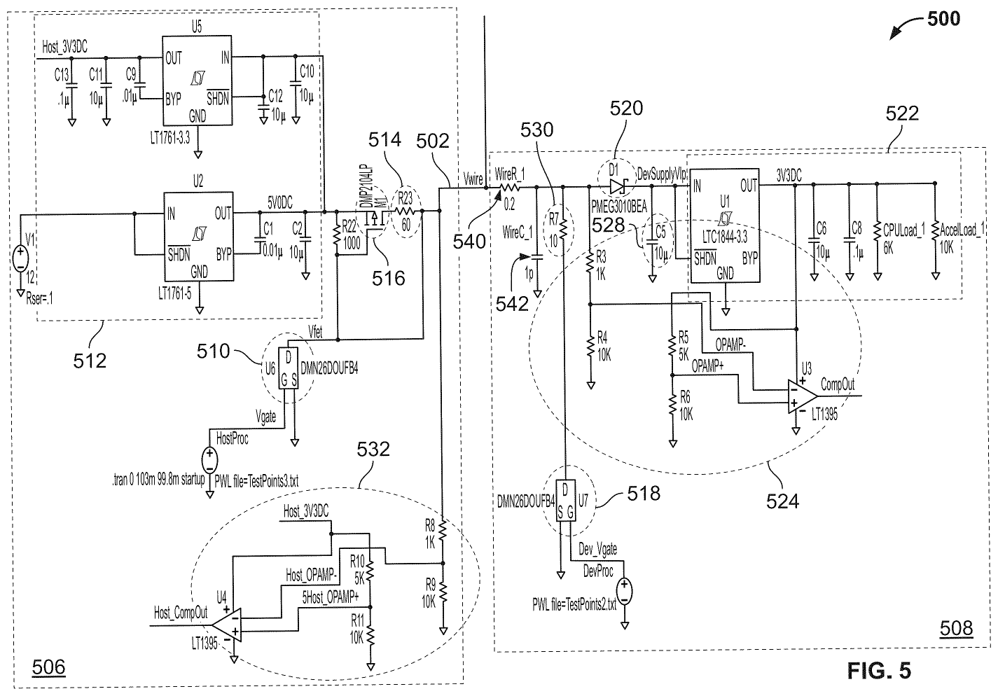

FIG. 5 illustrates an exemplary circuit 500 where the systems in accordance with some embodiments of the present specification were simulated. In one embodiment, a left side of the circuit 500 relates to a device 506 corresponding to first device 406 described with reference to FIG. 4. Similarly, a right side of circuit 500 relates to a device 508 corresponding to second device 408 of FIG. 4. A wire 502 (402) connects devices 506 (406) and 508 (408). Components of FIG. 5 correspond to the various components of FIG. 4 and are numbered similarly. For example, transistors 510 and 516 correspond to the transistors 410 and 416 of first device 406/506. In alternative embodiments, there can be multiple devices similar to device 508, which may be connected to device 506 through wire 502. In embodiments, the number of devices, similar to device 508, which may be connected through wire 502 to device 506, is limited by the power supplied by device 506 and consumed by multiple devices 508. Additionally, the numbers of multiple device 508 is limited by the protocols' ability to address more than a specific number of devices individually. In one embodiment, up to eight devices 508 are connected to device 506.

Referring again to FIG. 4, a voltage supply simulation ‘HostProc’ which is connected to V_{gate} simulates the interaction with processor 434. The remaining portions of processor 434 are not simulated. Similarly, a voltage supply simulation ‘DevProc’ simulates the interaction of the processor 426 and the remaining portions of that processor are not simulated. Referring to FIG. 5, a resistance ‘WIreR_1’ 540 and capacitance ‘WireC_1’ 542 are used to simulate parasitic resistances and capacitances of the wire 502. Finally, component U5 and associated components C9 through C13, in the power supply 512 of the first device 506, are optional. In some embodiments voltage dividers and op-amps make up comparator portions 524 and 532, respectively within devices 508 and 506, of circuit 500. However, there is no restriction to that portion of the circuit—other circuits such as those with internal references may also be used.

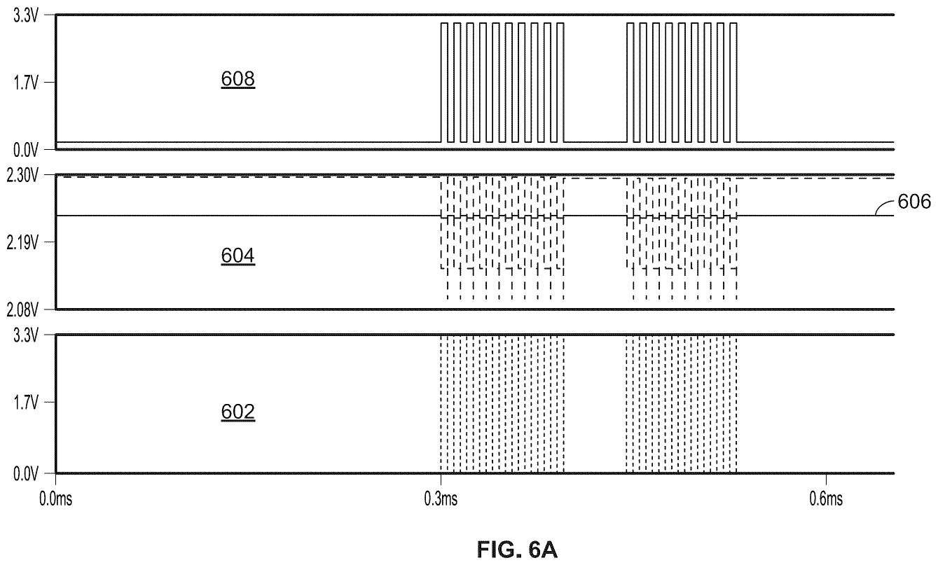

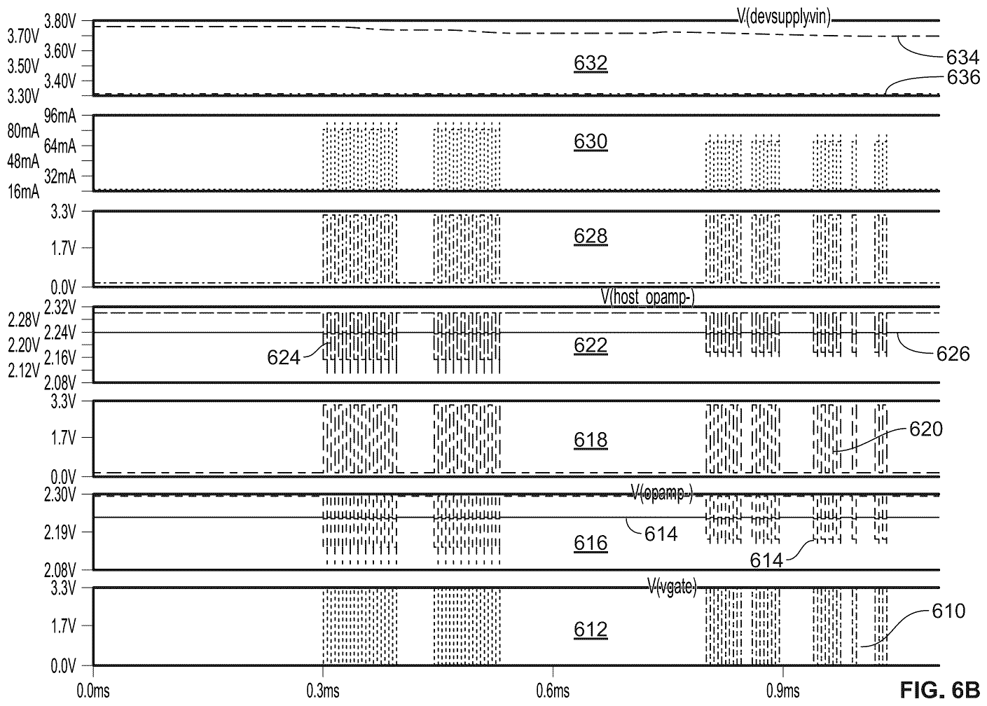

FIG. 6A illustrates transmission of data from first device 406/506 to second device 408/508, shown in FIGS. 4 and 5, in accordance with some embodiments of the present specification. Simultaneous reference to components of FIGS. 4 and 5 is made to enhance the description of the graphs. The figure shows transmission of a value of 0xFFFF (2 sets of 8 bits of 1's). Lower graph 602 shows the input provided to the transmit transistor (transmitting pin of transistor 410 of FIG. 4). Middle graph 604 shows comparator 424/524 op-amp inputs on second device 408/508 where a line 606 drawn across graph 604, is the reference voltage. In an embodiment, the reference voltage is between 2.19V and 2.3V. Top graph 608 shows the output of comparator 42/524 which is for all practical purposes identical to the transmitted input. In an exemplary embodiment, these transmissions were simulated at a rate of 100 KHz.

FIG. 6B illustrates transmission of data from second device 408/508 to first device 406/506 in response to the data transmission of FIG. 6A, in accordance with some embodiments of the present specification. In a first graph 632, a top trace 634 shows the input supply rail to the regulator 422 of second device 408/508. Some amount of expected sag is seen in trace 632, which occurs as a result of the power input intermittently dropping out. The output of that supply, however, remains constant at 3.3V (as shown in a lower trace 636 of graph 632) since the input is above the drop-out limit of the regulator. In some embodiments, regulators with low dropout values are selected to keep the slave device(s), such as second device 408/508, from drawing the power down below into the dropout range of their regulator(s). Alternatively, in some embodiments, the power resistor, such as resistor 414, is adjusted. A second graph 630 shows the current through resistor 414. A graph 628 below graph 630 shows comparator 432 output of first device 406/506, which is identical to the inputs from both of devices 406/506 and 408/508. A fourth graph 622 shows op-amp inputs 624 of first device 406/506 with a straight line 626 being the reference input voltage. A graph 618 shows an output 620 of the comparator 424. Traces 614 in a graph 616 show the signal and reference inputs, respectively, to the comparator 424. A trace 610 in a lower graph 612 is the input to transistor 418 of second device 408/508.

Additionally care needs to be taken to minimize the pulse widths of the bits transmitted and to allow enough recovery time in-between transmissions. Therefore, the pulse width of each bit is controlled. The bandwidth (baud rate) at which the transmission is driven may be a factor in controlling the pulse width for each bit. Independent of the baud rate, if the actual data transmission rate is low, a sag in the applied voltage is small. As the transmission rate increases and approaches the full bandwidth (baud rate) of the channel, the sag will worsen. In some embodiments, these signals are in the kHz range and the low portion of the pulse is minimized. In embodiments, pulse width depends upon the speed of each processor (434 and 426) and on the current draw of each 408 device. The size of capacitor 428, the leakage of diode 420, and the drop-out voltage value of regulator 422 may also affect the pulse width. Pulses of equal high and low widths that are between 1 kHz and 100 kHz are easily accommodated without resorting to larger or more expensive components.

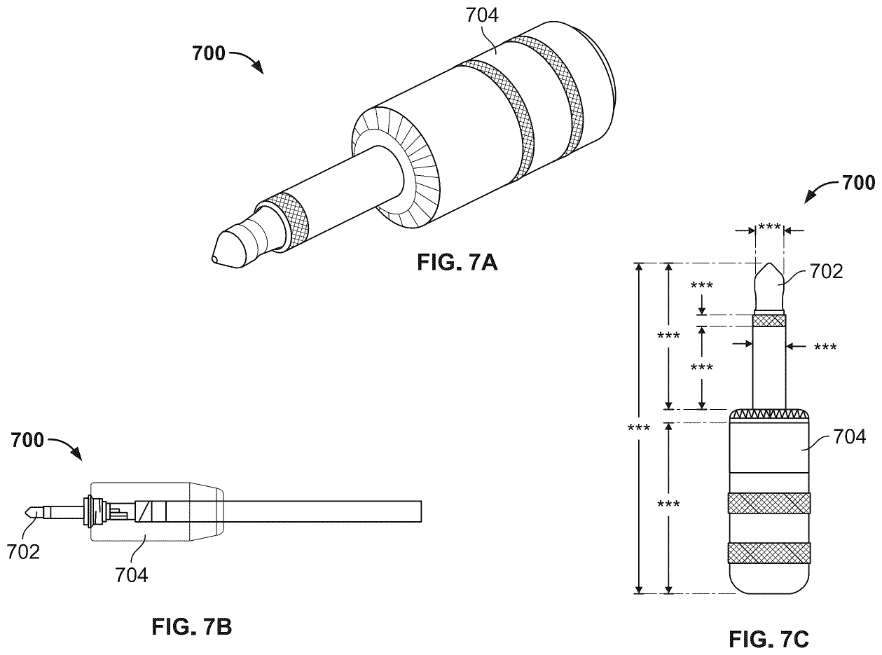

FIGS. 7A, 7B, and 7C illustrate images of a first end 700 of a wire that connects the first master device 406 (FIG. 4) and the second slave device 408 (FIG. 4), in accordance with some embodiments of the present specification. The first end 700 is a plug portion of the wire that connects with a physiological monitoring system, such as device 110 shown in FIG. 1. FIG. 7A illustrates a photograph of the plug portion 700, in accordance with some embodiments of the present specification. FIG. 7B illustrates a line drawing of the plug portion 700, including a cross-section view of its housing. FIG. 7C illustrates a line drawing of the plug portion 700 with dimensions of its components. Referring simultaneously to FIGS. 7A, 7B, and 7C, the plug portion comprises a pin 702 that is configured to be placed inside a corresponding female port of the physiological monitoring device, thereby providing an electrical connection between the wire and the physiological monitoring device. A pointed end of the pin 702, extending for a length of approximately 3.9 millimeters (mm) and with a diameter of approximately 2.35 mm, emerges from a band of approximately 0.8 mm that joins the pointed end of the pin 702 to an opposite end of the pin 702, which extends for a length of approximately 6.7 mm and has a diameter of approximately 2.46 mm. At the opposite end, the pin 702 is connected to a protective housing 704. The housing 704 encompasses a switch with electrical components of the wire that provides communication of power and data from and to the physiological monitoring device when the pin is plugged into the physiological monitoring device. A total length of the plug portion 700 extends for approximately 25.9 mm. In embodiments, the pin portion 700 plugs into a special pin on the physiological monitoring device or is built into a yoke.

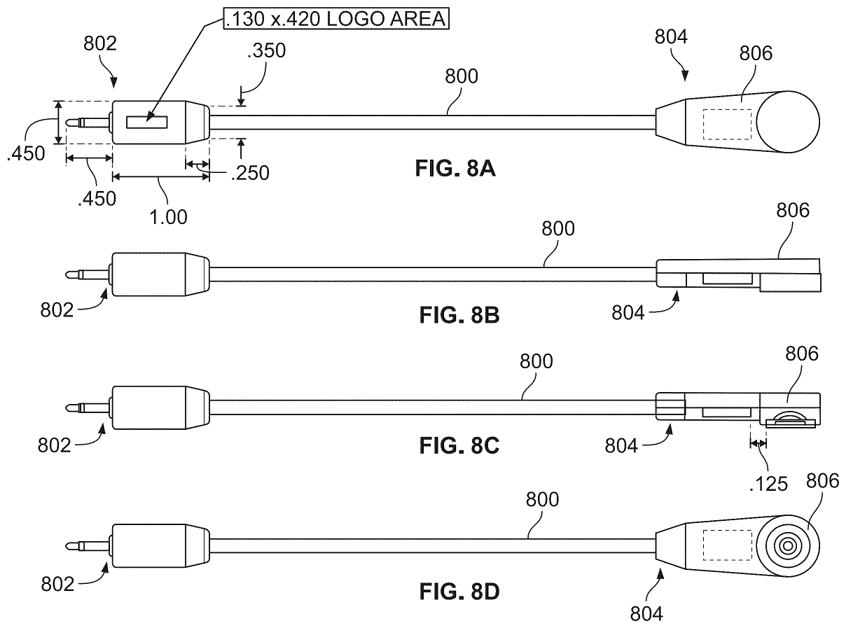

FIGS. 8A to 8D illustrate different views of a wire 800 with a plug portion 802 at one end and a receptacle portion 804 at the other end, in accordance with some embodiments of the present specification. FIG. 8A illustrates a top view of the receptacle portion 804 of the wire 800. FIG. 8B illustrates a side view of the receptacle portion 804 of the wire 800. FIG. 8C illustrates a cross-sectional side view of the receptacle portion 804 of the wire 800. FIG. 8D illustrates a bottom view of the receptacle portion 804 of the wire 800. In some embodiments, the wire 800 comprises a coaxial cable so as to avoid crosstalk. In different embodiments, the length of the wire 800 is different to suit various applications. For ECG applications, the length of wire 800 is similar to an ECG lead wire. Similarly, for ECG applications, wire 800 has a bend radius that is similar to the bend radius of an ECG lead wire. In embodiments, the wire 800 is applied and removed using its plug portion 802 using a similar force and technique for plugging and removing ECG lead wires. The receptacle portion 804 corresponds to the receptacle 200 of FIG. 2, which includes a housing 806 that encompasses components of an integrated motion sensor system. A lead connector is located at a one end of the connecting wire 800 within a portion of the housing 806 that interfaces with an electrode connector, analogous to an ECG electrode connector for the purpose of attaching receptacle portion 804 to the body of a patient. The motion sensor system, as described above, may comprise multiple components placed on a printed circuit board (PCB), and including elements that detect and process position and movement-related data. In embodiments, the housing 806 is watertight. The receptacle portion 804 may be configured as any of the known and used connectors to position on a subject.

The above examples are merely illustrative of the many applications of the system of present invention. Although only a few embodiments of the present invention have been described herein, it should be understood that the present invention might be embodied in many other specific forms without departing from the spirit or scope of the invention. Therefore, the present examples and embodiments are to be considered as illustrative and not restrictive, and the invention may be modified within the scope of the appended claims.

1-20. (canceled)

21. A system for monitoring at least one physiological parameter of a user concurrently with detecting motion of the user, the system comprising:
a first physiological parameter monitoring device comprising a first plurality of ports and a second port, wherein each port of the first plurality of ports is configured to physically couple to a proximal end of a first wire and wherein a distal end of the first wire comprises a sensor configured to monitor the at least one physiological parameter of the user; and
a motion detector adapted to physically attach to the user, wherein the motion detector comprises a second wire having a proximal end and a distal end, wherein the proximal end of the second wire is adapted to physically couple to the second port, wherein the distal end comprises a motion sensor, and wherein the motion sensor is adapted to receive power from the first physiological parameter monitoring device.

22. The system of claim 21, wherein the motion sensor is further configured to transfer data bi-directionally and asynchronously with the first physiological parameter monitoring device.

23. The system of claim 21, wherein the motion sensor is configured as a slave to the first physiological parameter monitoring device.

24. The system of claim 21, wherein the motion sensor is configured to acquire positional and movement information of the user and transmit the positional and movement information over the second wire to the first physiological parameter monitoring device.

25. The system of claim 21, wherein the at least one physiological parameter comprises at least one of ECG data, respiration data, SpO₂ data, and blood pressure data.

26. The system of claim 25, wherein the sensor is at least one of ECG sensor, respiration sensor, SpO₂ sensor, and blood pressure sensor.

27. The system of claim 21, wherein the motion sensor comprises a first printed circuit board and wherein the first printed circuit board is positioned in a receptacle adapted to attach to the user.

28. The system of claim 27, wherein the first printed circuit board is configured to operate as a slave to a master printed circuit board positioned in the first physiological parameter monitoring device.

29. The system of claim 28, wherein the first printed circuit board is configured to receive data from the master printed circuit board and compare said data to a reference value.

30. The system of claim 29, wherein the reference value is a voltage value.

31. The system of claim 21, wherein the motion sensor comprises a first printed circuit board having a power converter, a processor, a comparator and at least one of a three-axis accelerometer, a combination of a three-axis accelerometer and a gyroscope, or a combination of a three-axis accelerometer, a gyroscope, and a magnetometer.

32. A method for monitoring at least one physiological parameter of a user concurrently with detecting motion of the user, the method comprising:
physically coupling a proximal end of a first wire to at least one of a first plurality of ports of a first physiological parameter monitoring device comprising the first plurality of ports and a second port, wherein a distal end of the first wire comprises a sensor configured to monitor the at least one physiological parameter of the user;
physically attaching a motion detector to the user, wherein the motion detector comprises a second wire having a proximal end and a distal end, wherein the proximal end of the second wire is adapted to physically couple to the second port, wherein the distal end comprises a motion sensor, and wherein the motion sensor is adapted to receive power from the first physiological parameter monitoring device; and
acquiring the at least one physiological parameter generated by the user and the user's motion data by activating the first physiological parameter monitoring device.

33. The method of claim 32, further comprising transferring data bi-directionally and asynchronously between the motion sensor and the first physiological parameter monitoring device.

34. The method of claim 32, wherein the motion sensor is configured as a slave to the first physiological parameter monitoring device.

35. The method of claim 32, wherein acquiring the user's motion data comprises acquiring positional and movement information of the user by using the motion sensor and transmitting the positional and movement information over the second wire to the first physiological parameter monitoring device.

36. The method of claim 32, wherein acquiring the at least one physiological parameter generated by the user comprises acquiring at least one of the user's ECG data, respiration data, SpO₂ data, and blood pressure data.

37. The method of claim 32, wherein the sensor is at least one of ECG sensor, respiration sensor, SpO₂ sensor, and blood pressure sensor.

38. The method of claim 32, wherein the motion sensor comprises a first printed circuit board and wherein the first printed circuit board is positioned in a receptacle adapted to attach to the user.

39. The method of claim 38, further comprising operating the first printed circuit board as a slave to a master printed circuit board positioned in the first physiological parameter monitoring device.

40. The method of claim 39, further comprising receiving data from the master printed circuit board by the first printed circuit board and comparing said data to a reference value.

41. The method of claim 40, wherein the reference value is a voltage value.

42. The method of claim 41, wherein the motion sensor comprises a first printed circuit board having a power converter, a processor, a comparator and at least one of a three-axis accelerometer, a combination of a three-axis accelerometer and a gyroscope, or a combination of a three-axis accelerometer, a gyroscope, and a magnetometer.

43. The method of claim 32, further comprising determining a type and duration of one or more activities performed by the user from the user's motion data.

44. The method of claim 32, further comprising determining posture information of the user by using the acquired at least one physiological parameter of the user and the user's motion data to determine whether the user is in a sitting state, standing state, awake state, or asleep state.

45. The method of claim 32, further comprising determining levels of inactivity of the user by using the acquired at least one physiological parameter of the user and the user's motion data and generating an alarm if the user is required to be moved.

46. The method of claim 32, further comprising determining a fall of the user by using the acquired at least one physiological parameter of the user and the user's motion data.

47. The method of claim 32, further comprising determining movement by the user, wherein the movement is one of unwarranted, accidental, or unhealthy by using the acquired at least one physiological parameter of the user and the user's motion data.

48. The method of claim 32, further comprising determining whether the user is engaging in rapid movements, wherein the rapid movements is one or more of seizures, tremors, epileptic episodes, shivers, rapid breathing due to discomfort, coughing, vomiting, or rolling in bed by using the acquired at least one physiological parameter of the user and the user's motion data.

49. The method of claim 32, further comprising combining respiration detection data obtained from the acquired at least one physiological parameter of the user with the user's motion data for monitoring chest motion of the user and detecting apnea.

50. The method of claim 32, further comprising minimizing false ECG ST Segment alarms occurring due to positional changes of the user by using the acquired at least one physiological parameter of the user and the user's motion data.

51. The method of claim 32, further comprising minimizing false ECG rhythm alarms occurring due to positional changes of the user by using the acquired at least one physiological parameter of the user and the user's motion data.
